SEQUENCE LISTING (4 sequences)

SEQ ID NO: 1 - protein, 383 aa, Caenorhabditis elegans
MAFRHLAVARLKSLLVLCAVLLLVHAMIYKIPSLYENLTIGSSTLIADVDAMEAVLGNTASTSDDLLDTWNSTFSPISEVNQTSFMEDIRPILFPDNQTLQFCNQTPPHLVGPIRVFLDEPDFKTLEKIYPDTHAGGHGMPKDCVARHRVAIIVPYRDREAHLRIMLHNLHSLLAKQQLDYAIFIVEQVANQTFNRGKLMNVGYDVASRLYPWQCFIFHDVDLLPEDDRNLYTCPIQPRHMSVAIDKFNYKLPYSAIFGGISALTKDHLKKINGFSNDFWGWGGEDDDLATRTSMAGLKVSRYPTQIARYKMIKHSTEATNPVNKCRYKIMGQTKRRWTRDGLSNLKYKLVNLELKPLYTRAVVDLLEKDCRRELRRDFPTCF

SEQ ID NO: 2 - DNA, 1152 bp, Caenorhabditis elegans
atggcttttcgtcatttggcagtcgccagactcaagtcgttgctcgtactttgtgccgttcttctattagttcatgcaatgatttataagattccatcgctttacgagaaccttactatcggctcctcgacccttattgccgacgtcgacgcaatggaggcagtgctcgggaatacggcttccacttcggatgatctacttgatacgtggaattccacgttttcaccgatttctgaagttaatcagactagttttatggaggacattcgtccaatcctgttccccgacaaccagactcttcaattctgtaatcagacacctccccacctcgtcggacccatccgtgtattcctcgatgagcccgacttcaaaactctcgagaaaatctatccggacacgcacgccggtggacatggaatgcctaaggattgtgttgcaaggcatcgtgttgctattattgtgccctatagagatcgtgaagcacatttgagaataatgctccacaatttgcactcgttgctcgccaaacaacaattggactatgcaattttcattgtggagcaagtggcgaatcagacgtttaatcgcgggaaactaatgaacgttggatacgacgtagcatcacgcctctacccatggcagtgcttcatctttcatgatgtcgatttactgcccgaagatgaccgtaacctgtacacgtgtccaattcaaccacgtcatatgagtgtagcgatcgataaattcaattataaacttccatattcggcgatcttcggcggaatcagtgcactaacaaaagatcacctgaagaaaatcaatggattttcgaatgatttttggggttggggcggagaggacgacgatttggcgacgagaacatcgatggctggactgaaagtttcaagatatccgacacaaattgcacgatataaaatgattaagcactcgacggaagcgacgaatccagttaataaatgccgctacaaaataatgggccaaacgaagcgccgatggacacgtgacggcctaagcaatctgaagtataagctcgtaaatctggaattgaagcctctctacactcgagccgtcgtcgatttgctcgaaaaagactgccgccgggagctgcgaagggactttccaacgtgtttttag

SEQ ID NO: 3 - DNA, 26 bp, Artificial sequence
  Completely synthesized.
gccaccatggcttttcgtcatttggc

SEQ ID NO: 4 - DNA, 22 bp, Artificial sequence
  Completely synthesized.
ctaaaaacacgttggaaagtcc

(19) United States
(12) Patent Application Publication
(10) Pub. No.: US 2004/0086995 A1
(43) Pub. Date: May 6, 2004

(54) Beta1, 4-N-Acetylgalactosaminyltransferases, nucleic acids and methods of use thereof

(76) Inventors: Richard D. Cummings, Edmond, OK (US); Ziad S. Kawar, Oklahoma City, OK (US)

Correspondence Address: DUNLAP, CODDING & ROGERS P.C., PO BOX 16370, OKLAHOMA CITY, OK, 73113, US

(21) Appl. No.: 10/661,430

(22) Filed: Sep. 12, 2003

Related U.S. Application Data
(60) Provisional application No. 60/411,242, filed on Sep. 13, 2002.

(51) Int. Cl. C12N 9/10; C07H 21/04

(52) U.S. Cl. 435/193; 435/69.1; 435/320.1; 435/325; 536/23.2

(57) ABSTRACT

β1,4-N-Acetylgalactosaminyltransferases (β4GalNAcTs) and nucleic acids encoding the β4GalNAcTs or proteins having β4GalNAcT activity are described. The polynucleotides can be used to transform or transfect host cells for producing substantially pure forms of the enzyme, or for use in an expression system, or in vitro, for formation of a GalNAc β1,4 GlcNAc structure on proteins or peptides. Antibodies to the β4GalNAcTs and their use are also contemplated.

### CROSS-REFERENCE TO RELATED APPLICATIONS

This application claims the benefit under 35 U.S.C. § 119(e) of U.S. Provisional Application Serial No. 60/411,242, filed Sep. 13, 2002, entitled “β1,4-N-Acetylgalactosaminyltransferases and Methods Of Use”, the contents of which are expressly incorporated herein in their entirety by reference.

### STATEMENT REGARDING FEDERALLY FUNDED RESEARCH

Some aspects of this invention were made in the course of NIH Grant RO1 CH/HD54832-01; the U.S. Government has certain rights to this invention.

### BACKGROUND

The present invention is related to β1,4-N-Acetylgalactosaminyl transferases, and nucleic acids encoding the β1,4-N-Acetylgalactosaminyl transferases and to methods of use thereof.

Many of the functional moieties of complex glycoconjugates are in the terminal sequences of N- and O-glycans of glycoproteins and in glycolipids, which are recognized by a growing number of known carbohydrate binding proteins (1-4). A common terminal motif that is modified in a variety of ways by additions of other sugars and sulfate groups is the lactosamine sequence Galβ4GlcNAc-R, which is generated by a large family of β4galactosyltransferases (β4GalTs) acting on terminal GlcNAc residues (5). However, another common terminal motif found in vertebrate and invertebrate glycoconjugates is the GalNAcβ4GlcNAc-R (“LacdiNAc” or “LDN”) sequence. The LDN motif occurs in mammalian pituitary glycoprotein hormones, where the terminal GalNAc residues are 4-O-sulfated (6) and functions as a recognition marker for clearance by the endothelial cell Man/S4GGnM receptor (7). However, non-pituitary mammalian glycoproteins also contain LDN determinants (8-11) indicating that expression of LDN determinants in vertebrate glycoconjugates is more widespread than once thought. In addition, LDN and modifications of LDN sequences are common antigenic determinants in many parasitic nematodes and trematodes (12-17).

The LDN structure can be considered a variant of the more typical LacNAc structures generated by a family of UDPGal:GlcNAcβ-R β1,4Galactosyltransferases (β4GalT's) which includes the best characterized of all glycosyltransferases, the β4GalT I or lactose synthase (18-26). As more members of this family have been studied and the cDNAs encoding them cloned, it is evident that they share highly homologous regions within their amino acid sequences (27-35). These regions of homology are also found within the amino acid sequence of a snail UDP-GlcNAc:GlcNAcβ-R β1,4-N-acetylglucosaminyltransferases (β4GlcNAcT) (36,37). This latter finding raised the possibility that the β4GalNAcT enzyme(s) might also have amino acid sequence homology to members of the β4GalT family. Many studies have previously reported on the activity of an unidentified putative β4GalNAcT capable of generating LDN sequences (11, 38-41).

Although it appears that the lacNAc (LN) sequence Galβ4GlcNAc-R is a general terminal modification in vertebrate glycoconjugates, the LDN sequence also occurs in many vertebrate glycoproteins and glycolipids, including pituitary glycoprotein hormones (56) and many other glycoconjugates (8, 11, 57-59). A hormone-specific β4GalNAcT activity has been measured in the pituitary gland and other tissues which acts preferentially on glycoproteins containing a specific peptide motif (41, 56, 60-63). The GalNAc residue added to these hormones is subsequently 4-O-sulfated (64-66), and the resulting terminal GalNAc-4-SO₄ acts as a clearance signal that regulates their circulatory half-lives (6, 67-69). In addition to the hormone-specific β4GalNAcT, a motif-independent β4GalNAcT activity has been detected in extracts from many cells (62), including human 293 cells (11), bovine mammary gland (38), snails (70,71), insect cells (40), and schistosomes (39,72). The LDN motif is also a more common structural feature in invertebrate glycoconjugates compared to the LN motif, especially as seen in many parasitic nematodes and trematodes (12-17, 73). However, neither the enzyme(s) norgene(s) encoding the enzyme responsible for LDN synthesis have previously been defined.

As a result, there has remained a need in the field for complete identification of the gene (or genes) which encode the putative β4GalNAcTs responsible for the synthesis of LDN.

### BRIEF DESCRIPTION OF THE DRAWINGS

FIG. 1 depicts cDNA and a deduced protein sequence of Y73E7A.7 (Ceβ4GalNAcT). The putative transmembrane domain of the predicted protein encoded by Y73E7A.7 is double underlined; the Asp residues that are potentially N-glycosylated are in bold; and the DVD motifs are singly underlined.

FIG. 2 depicts the expression and purification of the protein encoded by Y73E7A.7 (SH-Ceβ4GalNAcT). (A) Intracellular (IC) extracts of wild-type CHO-Lec8 cells (Lec8) and CHO-Lec8 cells expressing a soluble, HPC4-epitope tagged protein encoded by Y73E7A.7 (SH-Ceβ4GalNAcT) (Lec8-GT) were tested for GalNAcT (gray bars) and GalT (hatched bars) activities using GlcNAcβ1-S-pNP as acceptor. The material captured by HPC4 beads from the extracellular medium (XC) from both cell types was also tested for these activities. The activity is indicated in pmol of donor sugar transferred per hour per 100,000 cells (IC) or 10 ml medium (XC). (B) Western blot using the HPC4 monoclonal antibody of the material captured on HPC4 beads from 10 ml of medium from Lec8-GT cells. The positions of molecular weight markers are indicated on the left in kDa.

FIG. 3 depicts HPAEC-PAD analysis of the reaction product catalyzed by SH-Ceβ4GalNAcT using GlcNAcβ1-O-pNP as acceptor. HPAEC of (A) GlcNAcβ1-O-pNP alone without incubation with Ceβ4GalNAcT and UDPGalNAc; (B) Ceβ4GalNAcT incubated with Ceβ4GalNAcT and UDPGalNAc. Standards are indicated as (a) GlcNAcβ1-4GlcNAcβ1-O-pNP; (b) GlcNAcβ1-3GalNAcα1-O-pNP (core 3-O-pNP); (c) GlcNAcβ1-6GalNAcα1-O-pNP (core 6-O-pNP); and (d) GlcNAcβ1-O-pNP.

FIG. 4 is a 400-MHz ¹H NMR spectrum of the reaction product catalyzed by SH-Ceβ4GalNAcT using GlcNAcβ1-S-pNP as acceptor.

FIG. 5 depicts the in vivo synthesis of LDN containing glycans. Western blots of cellular extracts of wild-type CHO-Lec8 cells (lane 1), CHO-Lec8 cells expressing SH-Ceβ4GalNAcT (lanes 2 and 3), wild-type CHO-Lec2 cells (lane 4), and CHO-Lec2 cells expressing SH-Ceβ4GalNAcT (lanes 5 and 6). The extracts in lanes 3 and 6 have been treated with N-glycanase. The membranes were probed with monoclonal antibodies against LDN (A) or the HPC4 tag (B). The positions of molecular weight markers are indicated on the left in kDa.

### SUMMARY OF THE INVENTION

According to the present invention, β1,4-N-Acetylgalactosaminyl transferases (β4GalNAcT), nucleic acids encoding β4GalNAcT, as well as methods for using same, is provided. Broadly, β4GalNAcT is required for the biosynthesis of animal cell glycoproteins. In one aspect, the invention also comprises homologous versions of β4GalNAcT proteins encoded by homologous cDNAs, vectors and host cells which express the homologous cDNAs, and methods of using the β4GalNAcT proteins and cDNAs.

In further aspects, the present invention contemplates cloning vectors which comprise the nucleic acids of the invention; and prokaryotic or eukaryotic expression vectors which comprise the nucleic acid molecules of the invention operatively associated with an expression control sequence. Accordingly, the invention further relates to a bacterial or eukaryotic cell transfected or transformed with an appropriate expression vector.

An object of the present invention is to provide a nucleic acid, in particular a DNA, that encodes a β4GalNAcT or a fragment thereof, or homologous derivatives or analogs thereof, or proteins having β4GalNAcT activity.

A further object of the present invention, while achieving the before-stated object, is to provide a cloning vector and an expression vector for such a nucleic acid molecule.

Yet another object of the present invention, while achieving the before-stated objects, is to provide a recombinant cell line that contains such an expression vector.

Yet a further object of the present invention, while achieving the before-stated objects, is to produce β4GalNAcT and/or fragments thereof.

A still further object of the present invention, while achieving the before-stated objects, is to provide methods for using β4GalNAcT and/or fragments thereof.

Other objects, features and advantages of the present invention will become apparent from the following detailed description when read in conjunction with the appended claims.

### DETAILED DESCRIPTION OF THE INVENTION

The LDN sequence, comprising of GalNAcβ1-4GlcNAc-R plus the by-product UDP are critical intermediates in the biosynthesis of certain animal cell glycoproteins. The LDN sequence is found in human and vertebrate glycoprotein hormones produced by the pituitary gland and is also found in a unique glycodelin, also known as placental protein, which has been implicated in endometriosis-related infertility. Further, LDN and its derivatives are major markers of glycoconjugates made by parasitic and non-parasitic invertebrates and may be implicated in host immune regulation and immune responses to infection. β4GalNAcT functions to synthesize the LDN sequence using specific acceptors in vitro as well as LDN sequences in animal cells.

In searching for the putative β4GalNAcT required for LDN synthesis, we examined genes in Caenorhabditis elegans. The C. elegans genome contains three open reading frames that encode proteins with sequence homology to the β4GalT family. One of these open reading frames (ORF R10E11.4; sqv-3) is predicted to encode a protein involved in vulval invagination (42), and is likely to be a UDPGal:Xyloseβ-R β1,4galactosyltransferases (32,43). Another of these open reading frames (ORF W02B12.11) encodes a protein for which no enzymatic activity has yet been reported. In the present invention, we identified and cloned a cDNA corresponding to a third open reading frame (ORFY73E7A.7) and demonstrated that it encodes a β4GalNAcT, which we have termed Ceβ4GalNAcT. The Ceβ4GalNAcT from C. elegans is active when expressed in mammalian cells in generating LDN determinants on N-glycans of glycoproteins.

As shown herein, a specific N-acetylgalactosaminyltransferase referred to herein as “Ceβ4GalNAcT” from C. elegans is capable of utilizing UDPGalNAc as the donor for the transfer of GalNAc residues to terminal GlcNAc acceptors in a wide variety of acceptors to generate the lacdiNAc (LDN) sequence GalNAcβ1,4GlcNAc-R. The enzyme is a member of the β4-galactosyltransferase family, although Ceβ4GalNAcT is unable to utilize UDPGal as the donor. In vertebrate cells, the recombinant form of Ceβ4GalNAcT is fully functional and capable of generating the LDN structure in complex-type N-glycans of glycoproteins. The present invention represents the first identification of a β4GalNAcT capable of generating the LDN sequence in animal glycoconjugates.

The polynucleotides of the present invention may be in the form of RNA or in the form of DNA, wherein the term “DNA” includes cDNA, genomic DNA and synthetic DNA. The DNA may be double-stranded or single-stranded, and if single-stranded, may be the coding strand or non-coding (anti-sense) strand. The coding sequence which encodes the mature polypeptide may be identical to the coding sequence shown herein or may be a different coding sequence which, as a result of the redundancy or degeneracy of the genetic code, encodes the same, mature polypeptide as the DNA coding sequences shown herein.

The polynucleotides which encode the mature polypeptides may include: only the coding sequence for the mature polypeptide; the coding sequence for the mature polypeptide and additional coding sequence such as a leader or secretory sequence or a proprotein sequence; the coding sequence for the mature polypeptide (and optionally additional coding sequence) and non-coding sequence, such as introns, or non-coding sequence 5′ and/or 3′ of the coding sequence for the mature polypeptide.

Thus, the term “polynucleotide encoding a polypeptide” encompasses a polynucleotide which includes only coding sequence for the polypeptide as well as a polynucleotide which includes additional coding and/or non-coding sequence.

The present invention further relates to variants of the hereinabove described polynucleotides which encode variants, fragments, analogs and derivatives of the polypeptide having the amino acid sequence of SEQ ID NO:1. The variants of the polynucleotide may be naturally occurring allelic variants of the polynucleotides or nonnaturally occurring variants of the polynucleotides.

Thus, the present invention includes polynucleotides encoding the same mature polypeptides as shown in SEQ ID NO:1, as well as variants of such polynucleotides which encode active variants, fragments, derivatives or analogs of said polypeptide. Such nucleotide variants include deletion variants, substitution variants and addition or insertion variants.

As hereinabove indicated, the polynucleotide may have a coding sequence which is a naturally occurring allelic variant of the coding sequences of SEQ ID NO:2. As is known in the art, an allelic variant is an alternate form of a polynucleotide sequence which may have a substitution, deletion or addition of one or more nucleotides which does not substantially adversely alter the function of the encoded polypeptide.

The present invention further relates to a β4GalNAcT polypeptide which has the amino acid sequence of SEQ ID NO:1 as well as active variants, fragments, analogs and derivatives of such polypeptide.

The terms “variant”, “fragment”, “derivative” and “analog” when referring to the polypeptide of SEQ ID NO:1, refer to β4GalNAcT which retains essentially the same or increased biological functions or activities as the native β4GalNAcT. Thus, an analog includes a proprotein which can be activated by cleavage of a proprotein portion to produce an active mature polypeptide. Fragments of β4GalNAcT include soluble, active proteins which have the N-terminal transmembrane region removed.

The polypeptide of the present invention may be a natural polypeptide or a synthetic polypeptide, or preferably a recombinant polypeptide.

The variant, fragment, derivative or analog of the polypeptide of SEQ ID NO:1 may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a proprotein sequence. Such variants, fragments, derivatives and analogs are deemed to be within the scope of one of ordinary skill in the art given the teachings herein.

The polypeptides and polynucleotides of the present invention are preferably provided in an isolated form, and preferably are purified substantially to homogeneity.

The term “isolated” means that the material is removed from its original environment (e.g., the natural environment if it is naturally occurring) in a form sufficient to be useful in performing its inherent enzymatic function. For example, a naturally-occurring polynucleotide or polypeptide present in a living animal is not isolated, but the same polynucleotide or polypeptide separated from some or all of the coexisting materials in the natural system, is isolated. Such polynucleotides could be part of a vector, and/or such polynucleotides or polypeptides could be part of a composition, and still be isolated in that such vector or composition is not part of its natural environment.

The present invention also relates to vectors which include polynucleotides of the present invention, host cells which are genetically engineered with vectors of the invention, and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, or a phage or other vectors known in the art. The engineered host cells can be cultured in conventional nutrient media modified as appropriate for activating promoters, selecting transformants or amplifying the β4GalNAcT genes. The culture conditions, such as temperature, pH and the like, are those previously used with the host cell selected for expression, and will be apparent to the ordinary skilled artisan.

The β4GalNAcT-encoding polynucleotides of the present invention may be employed for producing β4GalNAcT by recombinant techniques or synthetic in vitro techniques. Thus, for example, the β4GalNAcT-encoding polynucleotides may be included in any one of a variety of expression vectors for expressing the β4GalNAcT and/or any other desired proteins. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable in the host. In one embodiment, the additional protein desired to be expressed is P-selectin glycoprotein ligand-1 or a portion thereof or a synthetic peptide which has P-selectin binding activity.

The appropriate DNA sequence (or sequences) may be inserted into the vector by a variety of procedures. For example, the DNA sequence may be inserted into an appropriate restriction endonuclease sites(s) by procedures known in the art. Such procedures and others are deemed to be within the scope of a person of ordinary skill in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli lac or trp, the phage lambda PL promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells, such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate host to permit the host to express the protein as described elsewhere herein.

As representative examples of appropriate hosts, there may be mentioned: bacterial cells, such as E. coli, Streptomyces, Salmonella typhimurium; fungal cells, such as yeast; insect cells such as Drosophila and Sf9; animal cells such as CHO, COS, 293T or Bowes melanoma; plant cells, etc. The selection of an appropriate host is deemed to be within the scope of a person of ordinary skill in the art given the teachings herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. Bacterial: pQE70, pQE60, pQE-9 (Qiagen), pbs, pD10, phagescript, psiX174, pBluescript SK, pbsks, pNH8A, pNH16a, pNH18A, pNH46A (Stratagene); ptrc99a, pKK223-3, pKK233-3, pDR540, pRIT5 (Pharmacia). Eukaryotic: pWLNEO, pSV2CAT, pOG44, pXT1, pSG (Stratagene) pSVK3, pBPV, pMSG, pSVL (Pharmacia). However, any other plasmids or vectors may be used as long as they are replicable in the host.

Promoter regions can be selected from any desired gene using CAT (chloramphenicol transferase) vectors or other vectors with selectable markers. Two appropriate vectors are PKK232-8 and PCM7. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs. The host cells may be obtained using techniques known in the art. Suitable host cells include prokaryotic or lower or higher eukaryotic organisms or cell lines, for example bacterial, mammalian, yeast, or other fungi, viral, plant or insect cells. Methods for transforming or transfecting cells to express foreign DNA are well known in the art (See for example, U.S. Pat. No. 4,704,362; 76; U.S. Pat. No. 4,801,542; U.S. Pat. No. 4,766,075; and 77, all of which are incorporated herein by reference).

Introduction of the construct into the host cell can be effected by methods well known in the art such as by calcium phosphate transfection, DEAE-Dextran mediated 1transfection, or electroporation (78).

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by (77), the disclosure of which is hereby incorporated herein by reference.

Transcription of the DNA encoding the polypeptides of the present invention by higher eukaryotes may be increased by inserting an enhancer sequence into the vector. Enhancers are cis-acting elements of DNA, usually about from 10 to 300 bp that act on a promoter to increase its transcription. Examples include the SV40 enhancer, a cytomegalovirus early promoter enhancer, the polyoma enhancer, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracelluar medium. Optionally, the heterologous sequence can encode a fusion protein including an N-terminal or C-terminal identification peptide imparting desired characteristics, e.g., stabilization or simplified purification of expressed recombinant product.

Useful expression vectors for bacterial use are constructed by inserting one or more structural DNA sequences encoding one or more desired proteins together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacterial use can comprise a selectable marker and bacterial origin of replication derived from commercially available plasmids comprising genetic elements of the well known cloning vector pBR322, (ATCC 37017). These pBR322 “backbone” sections are combined with an appropriate promoter and the structural sequence to be expressed.

Following transformation of a suitable host strain and growth of the host strain to an appropriate cell density, the selected promoter is induced by appropriate methods (e.g., temperature shift or chemical induction) and cells are cultured for an additional period.

Cells are typically harvested by centrifugation, disrupted by physical or chemical methods, and the resulting crude extract retained for further purification. Microbial cells employed in expression of proteins can be disrupted by any convenient method, including freeze-thaw cycling, sonication, mechanical disruption, or use of cell lysing agents. Such methods are well known to a person of ordinary skill in the art.

Various mammalian cell culture systems can also be employed to express recombinant protein. Examples of mammalian expression systems include the COS-7 lines of monkey kidney fibroblasts, (79), and other cell lines capable of transcribing compatible vectors, for example, the C127, 293T, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice and polyadenylation sites may be used to provide the required nontranscribed genetic elements.

The β4GalNAcT polypeptides or portions thereof can be recovered and purified from recombinant cell cultures by methods including but not limited to ammonium sulfate or ethanol precipitation, acid extraction, anion or cation exchange chromatography, phosphocellulose chromatography, hydrophobic interaction chromatography, affinity chromatography, hydroxyl apatite chromatography, and lectin chromatography, alone or in combination. Protein refolding steps can be used as necessary in completing configuration of the mature protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

A recombinant β4GalNAcT of the invention, or functional variant, fragment, derivative or analog thereof, may be expressed chromosomally, after integration of the β4GalNAcT coding sequence by recombination. In this regard any of a number of amplification systems may be used to achieve high levels of stable gene expression (77).

The cell into which the recombinant vector comprising the nucleic acid encoding the β4GalNAcT is cultured in an appropriate cell culture medium under conditions that provide for expression of the β4GalNAcT by the cell. If full length β4GalNAcT is expressed, the expressed protein will comprise an integral transmembrane portion. If a β4GalNAcT lacking a transmembrane domain is expressed, the expressed soluble β4GalNAcT can then be recovered from the culture according to methods well known to persons of ordinary skill in the art. Such methods are described in detail, infra.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination.

The polypeptides, their variants, fragments or other derivatives, or analogs thereof, or cells expressing them can be used as an immunogen to produce antibodies thereto. These antibodies can be, for example, polyclonal or monoclonal antibodies. The present invention also includes chimeric, single chain, and humanized antibodies, as well as Fab (F(ab′)₂ fragments, or the product of an Fab expression library. Various procedures known in the art may be used for the production of such antibodies and fragments.

Antibodies generated against the polypeptides corresponding to a sequence of the present invention can be obtained by direct injection of the polypeptides into an animal or by other appropriate forms of administering the polypeptides to an animal, preferably a nonhuman. The antibody so obtained will then bind the polypeptide itself. In this manner, even a sequence encoding only a fragment of the polypeptide can be used to generate antibodies binding the whole native polypeptide. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (80), the trioma technique, the human B-cell hybridoma technique (81), and the EBV-hybridoma technique to produce human monoclonal antibodies (82).

Techniques described for the production of single chain antibodies (U.S. Pat. No. 4,946,778) can be adapted to produce single chain antibodies to immunogenic polypeptide products of this invention.

The polyclonal or monoclonal antibodies may be labeled with a detectable marker including various enzymes, fluorescent materials, luminescent materials and radioactive materials. Examples of suitable enzymes include horseradish peroxidase, alkaline phosphatase, β-galactosidase, or acetylcholinesterase; examples of suitable fluorescent materials include umbeliferone, fluorescein, fluorescein isothiocyanate, rhodamine, dichlorotriazinylamine fluorescein, dansyl chloride or phycoerythrin; examples of luminescent materials include luminol and aequorin; and examples of suitable radioactive material include S³⁵, Cu⁶⁴, Ga⁶⁷, Zr⁸⁹, Ru⁹⁷, Tc^{99m}, Rh¹⁰⁵, Pd¹⁰⁹, In¹¹¹, I¹²³, I¹²⁵, I¹³¹, Re¹⁸⁶, Au¹⁹⁸, Au¹⁹⁹, Pb²⁰³, At²¹¹, Pb²¹² and Bi²¹². The antibodies may also be labeled or conjugated to one partner of a ligand binding pair. Representative examples include avidin-biotin and riboflavin-riboflavin binding protein.

Methods for conjugating or labeling the antibodies discussed above with the representative labels set forth above may be readily accomplished using conventional techniques (such as described in U.S. Pat. No. 4,744,981; U.S. Pat. No., 5,106,951; U.S. Pat. No. 4,018,884; U.S. Pat. No. 4,897,255 U.S. Pat. No. 4,988,496; 83; and 84).

Due to the degeneracy of nucleotide coding sequences, other DNA sequences which encode substantially the same amino acid sequence as a β4GalNAcT gene described herein may be used in the practice of the present invention. These include but are not limited to nucleotide sequences comprising all or portions of β4GalNAcT genes which are altered by the substitution of different codons that encode the same amino acid residue within the sequence, thus producing a silent change. Likewise, the β4GalNAcT derivatives of the invention include, but are not limited to those containing, as a primary amino acid sequence, all or part of the amino acid sequence of the β4GalNAcT protein including altered sequences in which functionally equivalent amino acid residues are substituted for residues within the sequence, resulting in a conservative amino acid substitution. For example, one or more amino acid residues within the sequence can be substituted for another amino acid of a similar polarity, which acts as a functional equivalent. Substitutions for an amino acid within the sequence may be selected from, but are not limited to, other members of the class to which the amino acid belongs (See Table I).

TABLE I

| CLASS | AMINO ACID |
|---|---|
| Nonpolar: | Ala, Val, Leu, Ile, Pro, Met, Phe, |
| | Trp |
| Uncharged polar: | Gly, Ser, Thr, Cys, Tyr, Asn, Gln |
| Acidic: | Asp, Glu |
| Basic: | Lys, Arg, His |

Table I. Classes of amino acids suitable for conservative substitution.

As is well known to those skilled in the art, altering any given non-critical amino acid of a protein by conservative substitution may not significantly alter the activity of that protein because the side-chain of the amino acid which is inserted into the sequence may be able to form similar bonds and contacts as the side chain of the amino acid which has been substituted for. By “conservative substitution” is meant the substitution of an amino acid by another one of the same class; the classes according to Table I.

Non-conservative substitutions (outside the classes of Table I) are possible provided that these do not significantly diminish β4GalNAcT activity of the enzyme.

The polypeptides of the invention may be prepared synthetically, or more suitable, they are obtained using recombinant DNA technology. Thus, the invention further provides a nucleic acid which encodes any of the β4GalNAcT contemplated herein or any variants thereof which have enzymatic β4GalNAcT activity.

Such nucleic acids may be incorporated into an expression vector, such as a plasmid, under the control of a promoter as understood in the art. The vector may include other structures as conventional in the art, such as signal sequences, leader sequences and enhancers, and can be used to transform a host cell, for example a prokaryotic cell such as E. coli or a eukaryotic cell. Transformed cells can then be cultured and polypeptide of the invention recovered therefrom, either from the cells or from the culture medium, depending upon whether the desired product is secreted from the cell or not.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The genes encoding β4GalNAcT derivatives and analogs of the invention can be produced by various methods known in the art. The manipulations which result in their production can occur at the gene or protein level. For example, the cloned β4GalNAcT gene sequence can be modified by any of numerous strategies known in the art (77). The sequence can be cleaved at appropriate sites with restriction endonuclease(s), followed by further enzymatic modification if desired, isolated, and ligated in vitro. In the production of the gene encoding a derivative or analog of β4GalNAcT, care should be taken to ensure that the modified gene remains within the same translational reading frame as the β4GalNAcT coding sequence, uninterrupted by translation stop signals, in the gene region where the desired activity is encoded.

Within the context of the present invention, β4GalNAcT may include various structural forms of the primary protein which retain biological activity. For example, β4GalNAcT polypeptide may be in the form of acidic or basic salts or in neutral form. In addition, individual amino acid residues may be modified by oxidation or reduction. Furthermore, various substitutions, deletions or additions may be made to the amino acid or nucleic acid sequences, the net effect being that biological activity of β4GalNAcT is retained. Due to code degeneracy, for example, there may be considerable variation in nucleotide sequences encoding the same amino acid.

Mutations in nucleotide sequences constructed for expression of derivatives of β4GalNAcT polypeptide must preserve the reading frame phase of the coding sequences. Furthermore, the mutations will preferably not create complementary regions that could hybridize to produce secondary mRNA structures, such as loops or hairpins which could adversely affect translation of the mRNA.

Mutations may be introduced at particular loci by synthesizing oligonucleotides containing a mutant sequence, flanked by restriction sites enabling ligation to fragments of the native sequence. Following ligation, the resulting reconstructed sequence encodes a derivative having the desired amino acid insertion, substitution, or deletion.

Alternatively, oligonucleotide-directed site specific mutagenesis procedures may be employed to provide an altered gene having particular codons altered according to the substitution, deletion, or insertion required. Deletions or truncations of β4GalNAcT may also be constructed by utilizing convenient restriction endonuclease sites adjacent to the desired deletion. Subsequent to restriction, overhangs may be filled in, and the DNA religated. Exemplary methods of making the alterations set forth above (77).

As noted above, a nucleic acid sequence encoding a β4GalNAcT can be mutated in vitro or in vivo, to create and/or destroy translation, initiation, and/or termination sequences, or to create variations in coding regions and/or form new restriction endonuclease sites or destroy preexisting ones, to facilitate further in vitro or in vivo modification. Preferably, such mutations enhance the functional activity of the mutated β4GalNAcT gene product. Any technique for mutagenesis known in the art can be used, including but not limited to, in vitro site-directed mutagenesis (85; 86; 87; 88), use of TAB® linkers (Pharmacia), etc. PCR techniques are preferred for site directed mutagenesis (89).

It is well known in the art that some DNA sequences within a larger stretch of sequence are more important than others in determining functionality. A skilled artisan can test allowable variations in sequence, without expense of undue experimentation, by well-known mutagenic techniques (for example, see 90, 91, 92) by linker scanning mutagenesis (93), or by saturation mutagenesis (94). These variations may be determined by standard techniques in combination with assay methods described herein to enable those in the art to manipulate and bring into utility the functional units of upstream transcription activating sequence, promoter elements, structural genes, and polyadenylation signals. Using the methods described herein the skilled artisan can without application of undue experimentation test altered sequences within the upstream activator for retention of function. All such shortened or altered functional sequences of the activating element sequences described herein are within the scope of this invention.

The nucleic acid molecule of the invention also permits the identification and isolation, or synthesis of nucleotide sequences which may be used as primers to amplify a nucleic acid molecule of the invention, for example in the polymerase chain reaction (PCR) which is discussed in more detail below. The primers may be used to amplify the genomic DNA of other species which possess β4GalNAcT activity. The PCR amplified sequences can be examined to determine the relationship between the various β4GalNAcT genes.

The length and bases of the primers for use in the PCR are selected so that they will hybridize to different strands of the desired sequence and at relative positions along the sequence such that an extension product synthesized from one primer when it is separated from its template can serve as a template for extension of the other primer into a nucleic acid of defined length.

Primers which may be used in the invention are oligonucleotides of the nucleic acid molecule of the invention which occur naturally, as in purified products of restriction endonuclease digest, or are produced synthetically using techniques known in the art, such as phosphotriester and phosphodiesters methods (see for example, 95) or automated techniques (see for example, 96). The primers are capable of acting as a point of initiation of synthesis when placed under conditions which permit the synthesis of a primer extension product which is complementary to the DNA sequence of the invention i.e., in the presence of nucleotide substrates, an agent for polymerization, such as DNA polymerase, and at suitable temperature and pH. Preferably, the primers are sequences that do not form secondary structures by base pairing with other copies of the primer or sequences that form a hair pin configuration. The primer may be single or double-stranded. When the primer is double-stranded it may be treated to separate its strands before using to prepare amplification products. The primer preferably contains between about 7 and 50 nucleotides.

The primers may be labeled with detectable markers which allow for detection of the amplified products. Suitable detectable markers are radioactive markers such as P³², S³⁵, I¹²⁵, and H³, luminescent markers such as chemiluminescent markers, preferably luminol, and fluorescent markers, preferably dansyl chloride, fluorocein-5-isothiocyanate, and 4-fluor-7-nitrobenz-2-axa-1,3 diazole, enzyme markers such as horseradish peroxidase, alkaline phosphatase, β-galactosidase, acetylcholinesterase, or biotin.

It will be appreciated that the primers may contain non-complementary sequences provided that a sufficient amount of the primer contains a sequence which is complementary to a nucleic acid molecule of the invention or oligonucleotide sequence thereof which is to be amplified. Restriction site linkers may also be incorporated into the primers, allowing for digestion of the amplified products with the appropriate restriction enzymes facilitating cloning and sequencing of the amplified product.

In an embodiment of the invention a method of determining the presence of a nucleic acid molecule having a sequence encoding a β4GalNAcT, or a predetermined oligonucleotide fragment thereof in a sample, is provided comprising treating the sample with primers which are capable of amplifying the nucleic acid molecule or the predetermined oligonucleotide fragment thereof in a polymerase chain reaction to form amplified sequences, under conditions which permit the formation of amplified sequences, and assaying for amplified sequences.

The polymerase chain reaction refers to a process for amplifying a target nucleic acid sequence, (see for example 97, U.S. Pat. No. 4,863,195 and U.S. Pat. No. 4,683,202 which are incorporated herein by reference). Conditions for amplifying a nucleic acid template are described (98, which is also incorporated herein by reference).

It will be appreciated that other techniques such as the Ligase Chain Reaction (LCR) and NASBA may be used to amplify a nucleic acid molecule of the invention. In LCR, two primers which hybridize adjacent to each other on the target strand are ligated in the presence of the target strand to produce a complementary strand (99). NASBA is a continuous amplification method using two primers, one incorporating a promoter sequence recognized by an RNA polymerase and the second derived from the complementary sequence of the target sequence to the first primer (U.S. Pat. No. 5,130,238).

The present invention also provides novel fusion proteins in which any of the enzymes of the present invention are fused to a polypeptide such as protein A, streptavidin, fragments of c-myc, maltose binding protein, IgG, IgM, amino acid tag, etc. In addition, it is preferred that the polypeptide fused to the enzyme of the present invention is chosen to facilitate the release of the fusion protein from a prokaryotic cell or a eukaryotic cell, into the culture medium, and to enable its (affinity) purification and possibly immobilization on a solid phase matrix.

In another embodiment, the present invention provides novel DNA sequences which encode a fusion protein according to the present invention.

The present invention also provides novel immunoassays for the detection and/or quantitation of the present enzymes in a sample. The present immunoassays utilize one or more of the present monoclonal or polyclonal antibodies which specifically bind to the present enzymes. Preferably the present immunoassays utilize a monoclonal antibody. The present immunoassay may be a competitive assay, a sandwich assay, or a displacement assay, (see for example, 100) and may rely on the signal generated by a radiolabel, a chromophore, or an enzyme, such as horseradish peroxidase.

The invention will be more fully understood by reference to the following methods. However, the methods are merely intended to illustrate embodiments of the invention and are not to be construed to limit the scope of the invention.

Materials and Methods

All chemicals and reagents used in this study, unless otherwise indicated, were from Sigma (St. Louis, Mo.). The C. elegans cDNA library was a gift from Dr. Robert Barstead. The QIA Quick gel extraction kit was from Qiagen (Valencia, Calif.). Restriction enzymes were from New England Biolabs (Beverly, Mass.). The pCR 2.1 vector was from Invitrogen (Carlsbad, Calif.). The pcDNA3.1(+)-TH was a gift from Dr. Alireza R. Rezaie (Dept. of Biochemistry and Molecular Biology, St. Louis Univ. School of Medicine, St. Louis, Mo.). FuGENE 6 and Complete Protease Inhibitor Cocktail were from Roche (Indianapolis, Ind.). N-glycanase was from Glyko (Novato, Calif.). HighSignal West Pico Chemiluminescent Substrate was from Pierce (Rockford, Ill.). GlcNAcβ1-3GalNAcα1-O-pNP (core 3-O-pNP) and GlcNAcβ1-6GalNAcal-O-pNP (core 6-O-pNP) were obtained from Toronto Research Chemicals (Toronto, Canada).

Cloning and sequencing of the Ceβ4GalNAcT cDNA—A BlastP search of the NCBI non-redundant protein database for homologues of the human b4GalT I (accession # CAA39074) identified a hypothetical protein encoded by an open reading frame in the C. elegans genome designated Y73E7A.7. A cDNA was amplified by PCR from a mixed-stage C. elegans cDNA library using primers corresponding to the 5′ and 3′ ends of this open reading frame (5′-GCCACCATGGCTTTTCGTCATTTGGC-3′ (SEQ ID NO: 3); 5′-CTAAAAACACGTTGGAA AGTCC-3′) (SEQ ID NO: 4). Amplification was carried out at 95° C. for 2:30 min followed by 35 cycles at 95° C. for 50 sec, 53° C. for 50 s, and 72° C. for 1:50 min; then at 72° C. for 10 min. The PCR product was purified from an agarose gel slice using a QIA Quick gel extraction kit, cloned into the pCR 2.1 vector, and sequenced on both strands at the Sequencing Facility of the Oklahoma Medical Research Foundation (Oklahoma City, Okla.).

Construction of an expression vector encoding a soluble, epitope-tagged form of Ceβ4GalNAcT—A PsiI (partial)/PvuII DNA fragment starting at bp 87 of the Ceβ4GalNAcT open reading frame and extending beyond the stop codon was subcloned into the EcoRV site of the pcDNA 3.1(+)-TH vector. The resulting vector (pCMV-SH-Ceβ4GalNAcT) encodes a fusion protein, designated SH-Ceβ4GalNAcT, which consists of a signal peptide at the N-terminus followed by an HPC4 epitope then the catalytic domain of the Ceβ4GalNAcT (beginning at K34, the first amino acid after the transmembrane domain). This protein is under the transcriptional control of the CMV promoter, which is present in the vector.

Expression of SH-Ceβ4GalNAcT-CHO-Lec8 and CHO-Lec2 cells were transfected with pCMV-SH-Ceβ4GalNAcT using FuGENE 6, according to the manufacturer's instructions, and cultured in Dulbecco's Modified Eagle Medium containing 10% fetal calf serum and 600 mg/ml geneticin to select for stably transformed cells. After 4 weeks of culturing in medium containing geneticin, the cells were cultured in the same medium without geneticin, and the culture medium was harvested every 3 days and used to purify SH-Ceβ4GalNAcT. To assay intracellular b4GalNAcT activity and for Western blots, cells were washed with 75 mM sodium cacodylate pH 7.0 and lysed in a buffer of 50 mM sodium cacodylate pH 7.0, 20 mM MnCl₂, 1% Triton X-100, 1×Complete Protease Inhibitor Cocktail (EDTA-free). The lysates were centrifuged at 12,000×g for 3 min, and the supernatants were used for further analyses.

Purification of SH-C.E.β4GalNAcT—Medium containing SH-Ceβ4GalNAcT was centrifuged at 1,500×g for 5 min to remove cellular debris, and then incubated with HPC4-UltraLink beads (5 mg HPC4 antibody per ml of beads; 0.1 ml of beads per ml of medium) for one hour at room temperature on a rotating platform. The beads were collected by centrifugation at 600×g for 3 min, and washed three times with 10 ml of 100 mM sodium cacodylate pH 7.0, 2 mM CaCl₂. The beads were then resuspended in the same buffer with the addition of 20 mM MnCl₂, and used as the enzyme source. For Western blot analysis, the bound material was released by incubating the beads in a buffer of 50 mM sodium cacodylate pH 7.0, 20 mM EDTA for 10 min at room temperature, then collecting the supernatant.

SDS-PAGE and Western Blot analyses—Cell lysates were treated with N-glycanase in a buffer of 20 mM sodium phosphate pH 7.5, 50 mM b-mercaptoethanol, 0.1% SDS, 0.75% NP-40 for 3 h at 37° C. Control treatments were carried out in the same way, but without adding N-glycanase. The lysates were then mixed with loading buffer, resolved by SDS-PAGE (4-20% gradient), and transferred to a nitrocellulose membrane. The membrane was blocked with 5% BSA in a buffer of 20 mM Tris-HCl pH 7.2, 150 mM NaCl, 2 mM CaCl₂, 0.05% Tween 20 for 5 h at 4° C. It was then incubated with the primary antibody (mouse monoclonal anti-LDN IgM SMLDN1.1 (16), or HPC4 (IgG) in the same buffer (without BSA) for 1 h at room temperature; washed in the same buffer; and incubated with the secondary antibody (horseradish peroxidase-conjugated, goat anti-mouse IgM or IgG) as before. The membrane was then washed again; incubated in HighSignal West Pico Chemiluminescent Substrate for 2 min at room temperature; and exposed to a BioMax film (Kodak) for 1 min. The film was then developed using a processing machine (Konica SRX-101).

β4GalNAcT assays—Standard assays were performed essentially as described previously (40) in a 25 ml reaction mixture containing 2.5 mmol sodium cacodylate pH 7.2, 12.5 nmol UDP-[³H]GalNAc (2.5 Ci/mol), 1 mmol MnCl₂, 0.1 mmol ATP, 0.1 ml Triton X-100, 2 ml beads and acceptor substrate, containing 25 nmol of terminal GlcNAc at the non-reducing end unless otherwise indicated. Control assays lacking the acceptor substrate were carried out to correct for incorporation into endogenous acceptors, and all assays were carried out in duplicate. After incubation at 37° C. for 180 min the reaction was stopped. When oligosaccharides or glycopeptides were the acceptor, the labeled product was separated from unincorporated label by chromatography on a 1-ml column of Dowex 1-X8 (Cl⁻-form) according to Easton et al., (44). When oligosaccharide acceptors with hydrophobic aglycon (pNP) were used as the acceptor, the product was isolated using Sep-pak C-18 cartridges (Waters) as described (45). The isolated products were assayed for incorporation of radioactivity by liquid scintillation.

High-pH anion-exchange chromatography with pulsed amperometric detection (HPAEC-PAD)—The product catalyzed by SH-Ceβ4GalNAcT using GlcNAcβ1-O-pNP as acceptor was isolated using a Sep-pak C-18 cartridge (1 cc) and lyophilized. Three nmol of the product (dissolved in water) were analyzed by a Dionex HPAEC-PAD system, using a PA-1 column with a 100 mM NaOH solution at a flow rate of 1 ml per min. The standard containing the authentic LDN structure GalNAcβ1-4GlcNAcb1-O-pNP was synthesized using bovine β4GalT I and GlcNAcβ1-O-pNP as the acceptor for UDP-GlcNAc in the standard assay described above. Commercially acquired GlcNAcβ1-3GalNAcα1-O-pNP (core 3-O-pNP) and GlcNAcβ1-6GalNAcα1-O-pNP (core 6-O-pNP) were also used as standards.

Large scale synthesis of product for ¹H NMR analysis—Synthesis was carried out overnight at 37° C. in a 1 ml reaction mixture containing 50 mmol sodium cacodylate pH 7.0, 300 nmol GlcNAcβ1-S-pNP, 1 mmol UDPGalNAc, 20 mmol MnCl₂, 5 mmol ATP, 3 mmol NaN₃, and 100 ml beads. The product was then isolated using a Sep-pak C-18 cartridge (1 cc) and lyophilized.

400-Mz ¹H NMR—150 nmol of the product catalyzed by SH-Ceβ4GalNAcT using GlcNAcb1-S-pNP as acceptor were treated with D₂O.

Results

The results presented herein provide several new insights into the biosynthesis of animal cell glycoproteins. The Ceβ4GalNAcT we have identified in C. elegans is clearly a member of the β4GalT family of enzymes with some homology to those found in C. elegans to mammals. The enzyme responsible for LDN synthesis in animal cells has not been previously purified or well-characterized kinetically in a partially-purified form. Curiously, the GalT1 or lactose synthase is capable of utilizing both UDPGal and UDPGalNAc, and in the presence of a-lactalbumin, this enzyme is stimulated to utilize UDPGalNAc as the donor to generate LDN with free GlcNAc as the acceptor (74). Thus, it is possible that the LDN structure might not be generated by a separate enzyme specific for UDPGalNAc. Therefore, it is especially interesting that the Ceβ4GalNAcT, while a member of the b4GalT family, does not utilize UDPGal. The high homology in the protein sequence between Ceβ4GalNAcT and the β4GalT family members is not surprising, especially in light of a recent study on the effect of a point mutation on the donor sugar specificity of a β4GalT. That study demonstrated that changing a tyrosine residue (Y289) in the bovine β4GalT I to isoleucine altered its donor specificity from UDPGal to UDPGalNAc (21). It is noteworthy that the Ceβ4GalNAcT contains an isoleucine residue (1257) at the corresponding position.

Although the Ceβ4GalNAcT is able to act on most of the common types of mammalian N- and O-glycans, we have only a limited knowledge of the glycan structures produced in C. elegans. It has been reported that the LDN motif appears at the reducing end of O-glycans R-GalNAcβ4GlcNAc-Ser/Thr in unusual O-glycans of C. elegans (75). Whether the Ceβ4GalNAcT is responsible for synthesis of this type of structure is currently unknown.

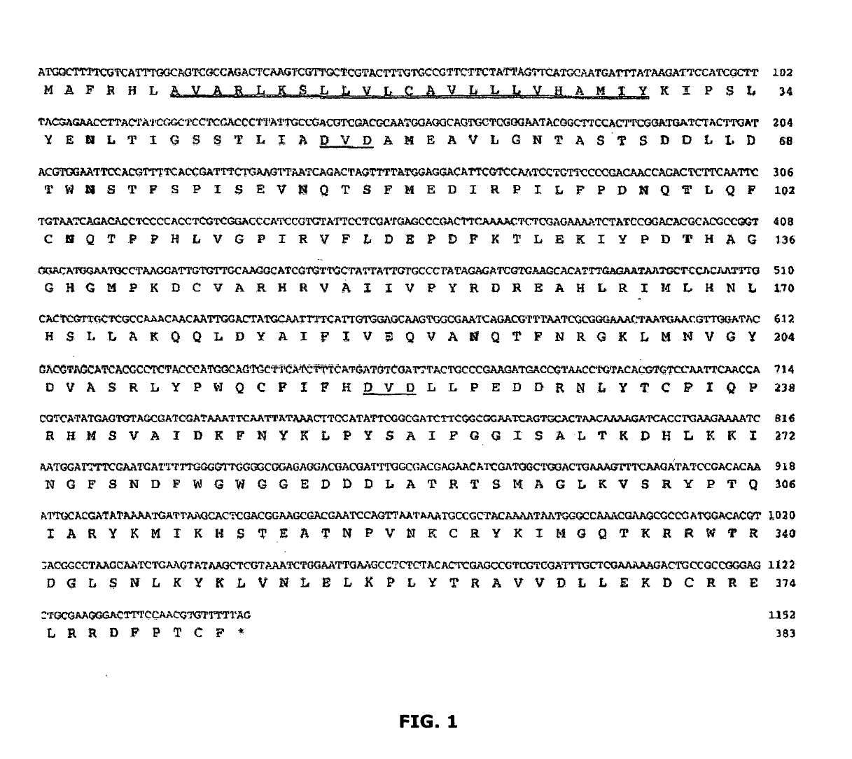

Isolation of the cDNA Encoded by Y73E7A.7 (Ceβ4GalNAcT)—A potential C. elegans open reading frame designated Y73E7A.7 was identified by a BlastP search as encoding a homologue of the human β4GalT I. An identical cDNA was amplified by PCR from a mixed-stage C. elegans cDNA library using primers corresponding to the 5′ and 3′ ends of this open reading frame, establishing that the gene is expressed in vivo. The cDNA of Y73E7A.7 encodes a predicted 383 amino acid protein with a single transmembrane domain in a type 2 topology. The protein is predicted to contain six potential N-glycosylation sites and two DVD motifs, which are thought to participate in metal ion binding (46) (FIG. 1). The protein sequence encoded by Y73E7A.7 is 35.5% identical to human β4GalT I, and is more closely related to the first four members of the β4GalT family (human β4GalT I, II, III, and IV) than to the others in that family (data not shown).

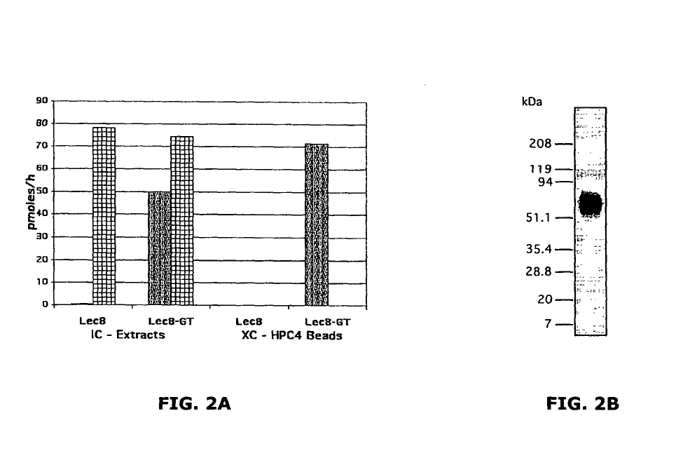

Expression and purification of a soluble, recombinant protein encoded by Y73E7A.7 (SH-Ceβ4GalNAcT)—To assess whether Y73E7A.7 encodes an active β4galactosyltransferase or possibly a β4N-acetylgalactosyltransferase, a soluble, recombinant form of the protein was generated lacking the cytoplasmic N-terminus and transmembrane domain and containing the 10-amino acid HPC4 peptide epitope at the new N-terminus. This construct was stably expressed in Chinese hamster ovary CHO-Lec8 cells. These cells are impaired in the transport of UDPGal into the Golgi (47) and consequently generate hybrid- and complex-type N-glycans containing terminal GlcNAc and O-glycans containing the simple Tn antigen GalNAcα1-Ser/Thr (48-50). The transfected cells expressing Y73E7A.7, but not the control mock transfected cells, acquired a novel intracellular GalNAcT activity in the cell extracts capable of utilizing UDPGalNAc as the donor and GlcNAcβ1-S-pNP as the acceptor (FIG. 2A). The recombinant protein containing the HPC4 epitope from extracellular medium was bound by HPC4-conjugated beads, confirming the β4GalNAcT activity of the enzyme encoded by the Y73E7A.7 (FIG. 2A). A Western blot of the material bound to the HPC4-conjugated beads confirmed that it corresponded to the predicted size of the HPC4-epitope tagged protein (FIG. 2B). These data demonstrate that Y73E7A.7 encodes an active β4GalNAcT and the enzyme was designated the C. elegans UDPGalNAc:GlcNAcb-R β1,4-N-acetylgalactosaminyltransferase (Ceβ4GalNAcT), and the soluble, HPC4-epitope tagged version was designated SH-Ceβ4GalNAcT.

Donor and substrate specificity of SH-Ceβ84GalNAcT—The enzyme purified from the medium using HPC4-conjugated beads was used in assays to further characterize its activity. In assays to determine its specificity for nucleotide-sugar donors (Table II), SH-CebβGalNAcT efficiently utilized UDPGalNAc, but did not significantly utilize UDPGal, UDPGlcNAc, or UDPGlc. In assays to determine its specificity for acceptor substrates (Table III), SH-Ceβ4GalNAcT efficiently utilized free GlcNAc and all substrates containing terminal β-linked GlcNAc in both N- and O-glycan type structures. SH-Ceβ4GalNAcT acted less effectively on α-linked GlcNAc or 6-sulfated GlcNAc, and did not significantly act on β-linked-Gal, -Glc, or -GalNAc acceptors. The acceptor substrate specificity of SH-Ceβ4GalNAcT is therefore similar to the broad specificity reported for human β4GalT I (31). In contrast, the snail β4-GlcNAcT has a marked preference for acceptors with β1,6-linked terminal GlcNAc (37) (see Table III for a side-by-side comparison).

In view of the sequence homology between Ceβ4GalNAcT and the β4GalT family, we examined whether the modifier protein a-lactalbumin would affect the acceptor specificity of SH-Ceβ4GalNAcT. α-Lactalbumin, which is expressed in lactating mammary glands, associates with β4GalT I and switches its acceptor specificity from R-GlcNAc to free Glc, thus forming lactose synthase (51). However, unlike its effect on β4GalT I, a-lactalbumin did not induce SH-Ceβ4GalNAcT to utilize Glc as an acceptor instead of GlcNAc (Table IV).

TABLE II
Sugar Nucleotide Specificity of the Ceb4GalNAcT.

| | | Relative activity |
|---|---|---|
| Acceptor | UDP-donor | (%)^{a} |
| GlcNAcβ-S-pNP | UDP-GalNAc | 100 |
| GlcNAcβ-S-pNP | UDP-GlcNAc | 0.7 |
| GlcNAcβ-S-pNP | UDP-Glc | 0.2 |
| GlcNAcβ-S-pNP | UDP-Gal | 1 |

^{a}says were carried out in duplicate as described in Experimental Procedures using SH-Ceβ4GalNAcT attached to HPC4-beads with a donor concentration of 0.5 mM and an acceptor concentration of 1 mM. For comparison, 100% activity corresponds to 5.9 nmol/min/ml beads suspension.

TABLE III
8/42 Acceptor Specificity of Ceβ4GalNAcT and Comparison to Other Members of the
β4GalT Family.

| | | Relative activity (%)^{a} | | |
|---|---|---|---|---|
| | | Ceβ4- | Human | L. stagnalis |
| Acceptor | | GalNacT | β4GalT I^{b} | β4GlcNAcT^{b} |
| 1. | GlcNAcβ-S-pNP | 285 | 232 | 5380 |
| 2. | GlcNAcα1-pNP | 14 | 39 | 95 |
| 3. | Galβ-pNP | 1 | | |
| 4. | Glcβ1-methyl-umbelliferone | 0.5 | | |
| 5. | GalNAcβ-pNP | 0.5 | | <10 |
| 6. | SO₄-6-GlcNAcβ1-pNP | 6 | 25 | |
| 7. | GlcNAcβ1-3GalNAcα-pNP | 145 | 197 | 250 |
| 8. | GlcNAcβ1-6(Galβ1-3)GalNAcα-pNP | 159 | 195 | 5570 |
| 9. | GlcNAc | 100 | 100 | 100 |
| 10. | GlcNAcβ1-3Gal | 121 | | 176 |
| 11. | GlcNAcβ1-6Gal | 328 | | 1590 |
| 12. | GlcNAcβ1-4GlcNAc+062 1-4GlcNAc | 115 | | 24 |
| 13. | GlcNAcβ1-6GlcNAc | 109 | | 467 |
| 14. | GlcNAcβ1-2Man | 132 | | 34 |
| 15. | GlcNAcβ1-6Man | 156 | | 425 |
| 16. | 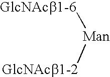 | 115 | | 176 |
| 17. | 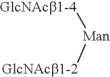 | 112 | | 58 |
| 18. | 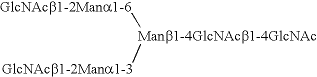 | 71 | 360 | |
| 19. | 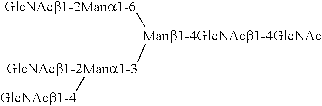 | 122 | 381 | |
| 20 | 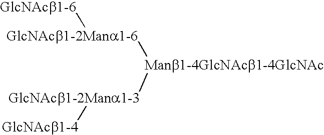 | 111 | 372 | |
| 21. | 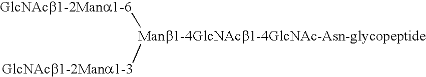 | 48 | 365 | |

^{a}Asays were carried out in duplicate as described in Experimental Procedures using SH-Ceβ4Ga1 NAcT attached to HPC4-beads with a donor concentration of 0.5 mM and an acceptor concentration of 1 mM terminal G1cNAc. For comparison, 100% activity (using free G1cNAc as acceptor) corresponds to 2.1 nmol/min/ml beads suspension.
^{b}Also for comparison, relative activities with the same acceptors for human β4GA1T I(32) and L. Stagnalis β4G1cNAcT (39) are taken from previous publications.

TABLE IV
Effect of α-Lactalbumin on Activity of the CeβGalAcT.

| | a-Lactalbumin | Relative activity |
|---|---|---|
| Acceptor | (5 mg/ml) | (%)^{a} |
| GlcNAc (1 mM) | − | 100 |
| GlcNAc (1 mM) | + | 40 |
| Glc (30 mM) | − | 3 |
| Glc (30 mM) | + | 6 |

^{a}Assays were carried out in duplicate as described in Experimental Procedures using SH-Ceβ4GalNAcT attached to HPC4-beads with a UDPGalNAc concentration of 0.5 mM. For comparison, the 100% activity corresponds to 2.1 nmol/min/ml beads suspension.

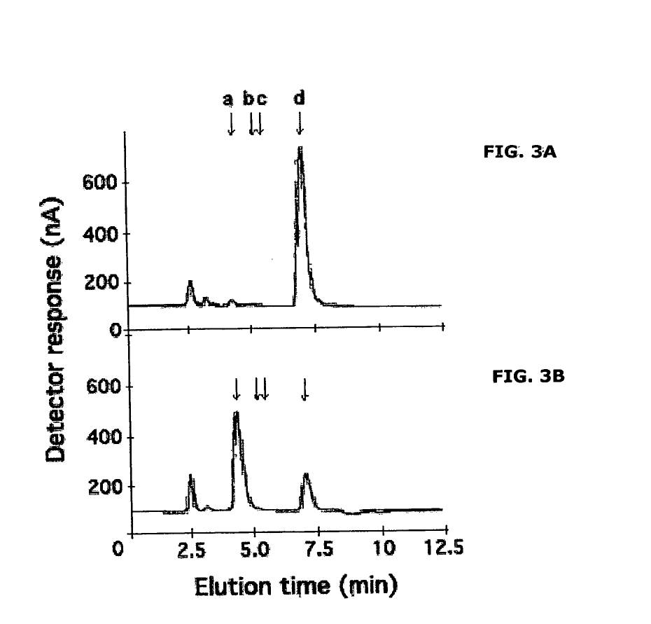

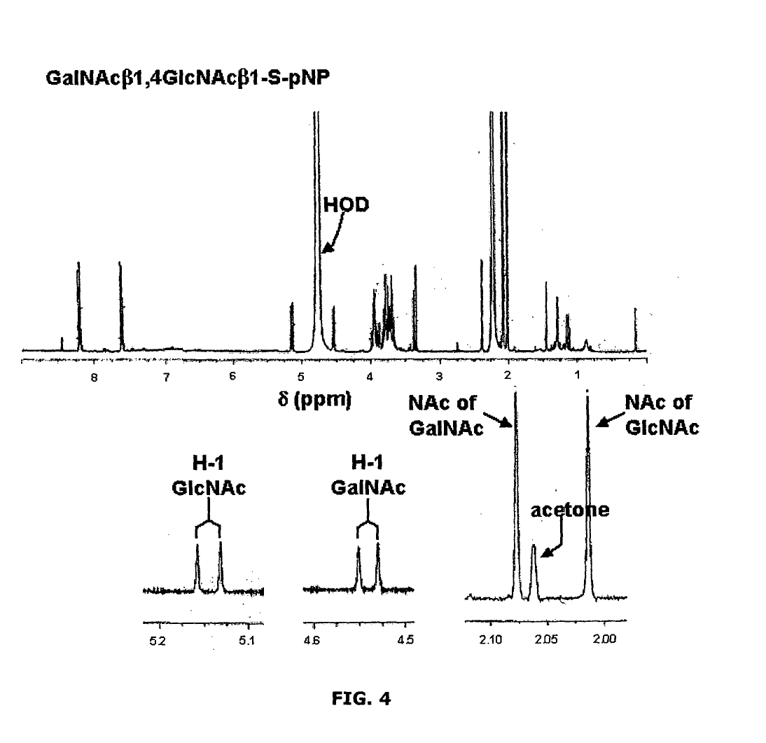

Product characterization by HPAEC-PAD and ¹H NMR—The product generated by SH-Ceβ4GalNAcT using GlcNAcβ1-O-pNP as acceptor was analyzed by HPAEC-PAD (FIG. 3). The product co-eluted with the authentic GalNAcβ1-4GlcNAcβ1-O-pNP standard, but not with two other disaccharide-O-pNP standards (GlcNAcβ1-3GalNAcα1-O-pNP and GlcNAcβ1-6GalNAcα1-O-pNP). To further establish the structure of the product generated by SH-Ceβ4GalNAcT using GlcNAcβ1-S-pNP as acceptor, the product was analyzed by ¹H NMR spectroscopy (FIG. 4). The spectrum shows two H-1 doublets at d=5.146 ppm and 4.540 ppm. The coupling constants of the H-1 doublets (10.5 Hz and 8.5 Hz, respectively) indicate that both C-1 atoms are in b-anomeric conformation (52). The doublet at 5.146 ppm and the signal at d=2.013 ppm can be assigned to the H-1 and the CH₃-NAc of GlcNAcβ1-S-pNP by analogy to the resonance positions in GlcNAcβ1-4GlcNAcβ1-S-pNP (36). The doublet at d=4.540 ppm and the signal at d=2.077 ppm have shifts that are close to those reported for a β4-linked GalNAc residue (39,40). The NMR spectrum therefore confirms that the analyzed product is GalNAcβ1-4GlcNAcβ1-S-pNP.

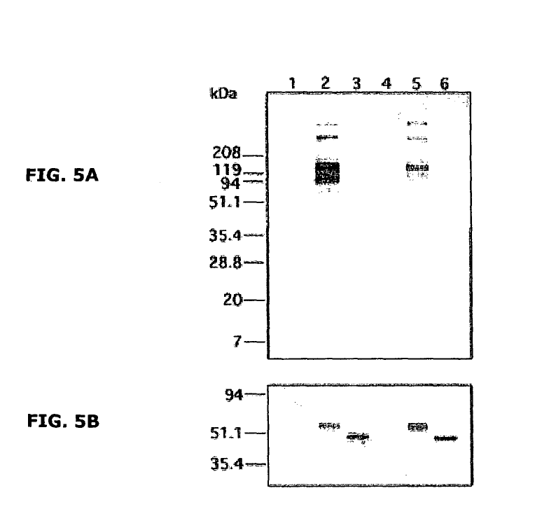

In vivo synthesis of LDN structures on N-glycans by SH-Ceβ4GalNAcT—Since SH-Ceβ4GalNAcT was active in cell extracts when expressed in CHO-Lec8 cells (FIG. 1), we examined whether it would act to produce LDN structures on endogenous glycan acceptors. Cell lysates from non-transfected CHO-Lec8 and CHO-Lec2 cells and transfected CHO-Lec8 and CHO-Lec2 cells expressing SH-Ceβ4GalNAcT were examined for the presence of LDN determinants by a Western blot analysis using a monoclonal antibody SMLDN1.1 against LDN (16) (FIG. 5). As indicated above the CHO-Lec8 cells are deficient in UDPGal transport into the Golgi (47), whereas the CHO-Lec2 cells are deficient in CMPSialic acid transport into the Golgi, and hence generate non-sialylated glycans terminating in Gal residues (53). Non-transfected CHO-Lec8 and CHO-Lec2 cells did not express detectable levels of LDN determinants as detected by SMLDN1.1. In contrast, both cell lines expressing SH-Ceβ4GalNAcT expressed the LDN epitope on several glycoproteins. Transfected CHO-Lec2 cells expressed lower levels of LDN determinants than transfected CHO-Lec8, possibly due to competition from endogenous β4GalTs. It would be predicted that the Ceβ4GalNAcT might only add GalNAc to N-glycans in CHO cells, since CHO cells produce O-glycans of the core 1 structure (Galβ3GalNAcα1Ser/Thr) lacking in GlcNAc residues (54,55). Cell extracts derived from CHO cell lines transfected with cDNA encoding Ceβ4GalNAcT were treated with N-glycanase to determine whether LDN determinants were present in N-glycans. N-glycanase treatment quantitatively removed the LDN-reactive epitopes from glycoproteins, demonstrating that LDN was expressed on N-glycans by the SH-Ceβ4GalNAcT.

It will be appreciated that the invention includes nucleotide or amino acid sequences which have substantial sequence homology (identity) with the nucleotide and amino acid sequences shown in the Sequence Listings. The term “sequences having substantial sequence homology” includes those nucleotide and amino acid sequences which have slight or inconsequential sequence variations from the sequences disclosed in the Sequence Listings, i.e. the homologous sequences function in substantially the same manner to produce substantially the same polypeptides as the actual sequences. The variations may be attributable to local mutations or structural modifications.

Substantially homologous (identical) sequences further include sequences having at least 90% sequence homology (identity) with the β4GalNAcT polynucleotide or polypeptide sequences shown herein or other percentages as defined elsewhere herein.

As noted elsewhere herein, the present invention includes the polynucleotide sequence SEQ ID NO:2 and coding sequences thereof which encode SEQ ID NO:1 or active portions thereof.

The polynucleotide may comprise untranslated regions upstream and/or downstream of the coding sequence and a coding sequence (which by convention includes the stop codon).

The term “identity” or “homology” used herein is defined by the output called “Percent Identity” of a computer alignment program called ClustalW, a program component of MacVector Version 6.5 by the Genetics Computer Group at University Research Park, 575 Science Dr., Madison, Wis. 53711. “Similarity” values provided herein are also provided as an output of the ClustalW program using the alignment values provided below. As noted, this program is a component of widely used package of sequence alignment and analysis programs called MacVector Version 6.5, Genetics Computer Group (GCG), Madison, Wis. The ClustalW program has two alignment variables, the gap creation penalty and the gap extension penalty, which can be modified to alter the stringency of a nucleotide and/or amino acid alignment produced by the program. The settings for open gap penalty and extend gap penalty used herein to define identity for amino acid alignments were as follows:

Open Gap penalty=10.0

Extend Gap penalty=0.05

Delay Divergent=40%

The program used the BLOSUM series scoring matrix. Other parameter values used in the percent identity determination were default values previously established for the 6.5 version of the ClustalW program (101).

In general, polynucleotides which encode β4GalNAcT are contemplated by the present invention. In particular, the present invention contemplates the DNA sequence SEQ ID NO: 2 and coding portions thereof, and portions of said sequences which encode soluble forms of β4GalNAcT, that is, β4GalNAcT lacking a transmembrane domain.

The invention further contemplates polynucleotides which are at least about 50% homologous, 60% homologous, 70% homologous, 80% homologous or 90% homologous to the coding sequence SEQ ID NO:2, where homology is defined as strict base identity, wherein said polynucleotides encode proteins having β4GalNAcT activity.

The present invention further contemplates nucleic acid sequences which differ in the codon sequence from the nucleic acids defined herein due to the degeneracy of the genetic code, which allows different nucleic acid sequences to code for the same protein as is further explained herein above and as is well known in the art. The polynucleotides contemplated herein may be DNA or RNA. The invention further comprises DNA or RNA nucleic acid sequences which are complementary to the sequences described above.

The present invention further comprises polypeptides which are encoded by the polynucleotide sequences described above. In particular, the present invention contemplates polypeptides having β4GalNAcT activity including SEQ ID NO: 1 and variants thereof which lack the transmembrane domain and which are therefore soluble. The present invention further contemplates polypeptides which differ in amino acid sequence from the polypeptides defined herein by substitution with functionally equivalent amino acids, resulting in what are known in the art as conservative substitutions, as discussed above herein.

Also included in the invention are polynucleotide sequences which hybridize to the polynucleotide set forth in SEQ ID NO:2 or coding sequences thereof, under stringent or relaxed conditions (as well known to persons of ordinary skill in the art), and which encode proteins having β4GalNAcT activity.

Hybridization and washing conditions are well known. (See 77, particularly Chapter 11 and Table 11.1 therein (expressly entirely incorporated herein by reference). The conditions of temperature and ionic strength determine the “stringency” of the hybridization.

In one embodiment, high stringency conditions are prehybridization and hybridization at 68° C., washing twice with 0.1×SSC, 0.1% SDS for 20 minutes at 22° C. and twice with 0.1×SSC, 0.1% SDS for 20 minutes at 50° C. Hybridization is preferably overnight.

In another embodiment, low stringency conditions are prehybridization and hybridization at 68° C., washing twice with 2×SSC, 0.1% SDS for 5 minutes at 22° C., and twice with 0.2×SSC, 0.1% SDS for 5 minutes at 22° C. Hybridization is preferably overnight.

In an alternative embodiment, very low to very high stringency conditions are defined as prehybridization and hybridization at 42° C. in 5×SSPE, 0.3% SDS, 200 ug/ml sheared and denatured salmon sperm DNA, and either 25% formamide for very low and low stringencies, 35% formamide for medium and medium-high stringencies, or 50% formamide for high and very high stringencies, following standard Southern blotting procedures.

The carrier material is then washed three times each for 15 minutes using 2×SSC, 0.2% SDS preferably at least 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

It is well known in the art that numerous equivalent conditions may be employed which comprise low stringency conditions; (e.g., factors such as the length and nature) (e.g., base composition) of the probe and nature of the target (e.g., base composition, present in solution or immobilized,), and the concentration of the salts and other components (e.g., the presence or absence of formamide, dextran sulfate, polyethylene glycol) are considered as such and the hybridization solution may be varied to generate conditions of low stringency hybridization different form, but equivalent to, the above listed conditions. In addition, conditions which promote hybridization under conditions of high stringency (e.g., increasing the temperature of the hybridization and/or wash steps, the use of formamide in the hybridization solution) are also known in the art.

When used in reference to a double-stranded nucleic acid sequence such as a cDNA or genomic clone, the term “substantially homologous” refers to any probe which can hybridize to either or both strands of the double-stranded nucleic acid sequence under conditions of low stringency as described above.

When used in reference to a single-stranded nucleic acid sequence, the term “substantially homologous” refers to any probe which can hybridize (i.e., it is the complement of) the single-stranded nucleic acid sequence under conditions of low stringency as described above.

As used herein, the term “hybridization” is used in reference to the pairing of complementarity nucleic acids. Hybridization and the strength of hybridization (i.e., the strength of the association between the nucleic acids) is impacted by such factors as the degree of complementary between the nucleic acids, stringency of the conditions involved, the Tₘ (melting temperature) of the formed hybrid, and the G:C ratio within the nucleic acids.

As used herein the term “stringency” is used in reference to the conditions of temperature, ionic strength, and the presence of other compounds such as organic solvents, under which nucleic acid hybridizations are conducted.

As used herein, the terms “cell,” “cell line,” and “cell culture” are used interchangeably and all such designations include progeny. The words “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

As used herein, the term “vector” is used in reference to nucleic acid molecules that transfer DNA segment(s) from one cell to another. The term “vehicle” is sometimes used interchangeably with “vector”.

The terms “recombinant DNA vector” as used herein refers to DNA sequences containing a desired coding sequence and appropriate DNA sequences necessary for the expression of the operably linked coding sequence in a particular host organism. DNA sequences necessary for expression in prokaryotes include a promoter, optionally and operator sequence, a ribosome binding site and possibly other sequences. Eukaryotic cells are known to utilize promoters, polyadenylation signals and enhancers. It is not intended that the term be limited to any particular type of vector. Rather, it is intended that the term encompass vectors that remain autonomous within host cells (e.g., plasmids), as well as vectors that result in the integration of foreign (e.g., recombinant nucleic acid sequences) into the genome of the host cell.

The terms “expression vector” or “recombinant expression vector” as used herein refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. It is contemplated that the present invention encompasses expression vectors that are integrated into host cell genomes, as well as vectors that remain unintegrated into the host genome.

The terms “in operable combination,” “in operable order,” and “operably linked,” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The proteins described herein may be expressed in either prokaryotic or eukaryotic host cells. Nucleic acid encoding the proteins may be introduced into bacterial host cells by a number of means including transformation or transfection of bacterial cells made competent for transformation by treatment with calcium chloride or by electroporation. If the proteins are to be expressed in eukaryotic host cells, nucleic acid encoding the protein may be introduced into eukaryotic host cells by a number of means including calcium phosphate co-precipitation, spheroplast fusion, electroporation, microinjection, lipofection, protoplast fusion, and retroviral infection, for example. When the eukaryotic host cell is a yeast cell, transformation may be affected by treatment of the host cells with lithium acetate or by electroporation, for example.

Utility

As noted above, the availability of the β4GalNAcT contemplated herein will be a valuable tool for the in vitro and in vivo synthesis of glycans comprising LDN structures, especially for the production of antigenic glycans and pharmaceutical or commercial products containing LDN structures.

The present invention may comprise variants of Ceβ4GalNAcT, wherein the variant is characterized as a protein having at least 25% of the enzyme activity of Ceβ4GalNAcT, at least 50% of the activity of Ceβ4GalNAcT, at least 75% of the activity of Ceβ4GalNAcT, at least 100% of the activity of Ceβ4GalNAcT, or greater than 100% of the activity of Ceβ4GalNAcT, as measured by assays described herein.

In a preferred version of the invention, the invention comprises a recombinant, 4-N-acetylgalactosaminyl-transferase for synthesizing LDN determinants in vitro or in vivo, or a gene for synthesizing the β4GalNAcT, or a vector or host cell comprising the gene.

In particular, the β4GalNAcTs (UDPGalNAc:GlcNAcβ-R β1,4-N-acetylgalactosaminyltransferase) described and contemplated herein can be used to generate LDN sequences in cultured animal cells, or in transgenically-engineered animals. It can be used to generate the LDN sequence on recombinant glycoprotein co-expressed with the β4GalNAcT in animal cells or non-vertebrate host cells or transgenically-engineered animals. It can be used in vitro to generate the LDN structure on monosaccharide acceptors or their derivatives and on simple or complex oligosaccharide acceptors. The β4GalNAcT of the present invention can be used to generate LDN containing material for production of vaccine derivatives for prevention and/or treatment of infectious diseases caused by organisms carrying the LDN structure or its derivatives. The gene encoding the β4GalNAcT can be used to screen for the predicted presence of RNA transcripts encoding the enzyme in human and animal tissues. The gene encoding the β4GalNAcT could be used to identify homologs of this gene in vertebrate or invertebrate cells. The gene encoding the β4GalNAcT when transposed or transfected into a cell could be used to generate a recombinant form of the β4GalNAcT for use as an enzyme in vitro or to generate antibodies to the protein for use in detection and/or treatment of infectious diseases or in studying expression of the enzyme. The recombinant β4GalNAcT can be used to generate antibodies to itself, as described below.

The present invention contemplates monoclonal or polyclonal antibodies raised against β4GalNAcT or active variants thereof. The antibody may be prepared by a method comprising immunizing a suitable animal or animal cell with β4GalNAcT, an active variant thereof, or any immunogenic portion thereof to obtain cells for producing an antibody to said mutant, fusing cells producing the antibody with cells of a suitable cell line, and selecting and cloning the resulting cells producing said antibody, or immortalizing an unfused cell line producing said antibody, e.g., by viral transformation, followed by growing the cells in a suitable medium to produce said antibody and harvesting the antibody from the growth medium in a manner well known to those of ordinary skill in the art. The recovery of the polyclonal or monoclonal antibodies may be preformed by conventional procedures well known in the art. (see, for example, 80).

Antisera containing antibodies of the invention are readily prepared by injecting a host animal (e.g., a mouse, pig or rabbit) with a protein of the invention and then isolating serum from it after a waiting suitable period for antibody production, e.g., 14 to 28 days. Antibodies may be isolated from the blood of the animal or its sera by use of any suitable known method, e.g., by affinity chomatography using immobilized mutants of the invention or the mutants they are conjugated to, e.g., GST, to retain the antibodies. Similarly monoclonal antibodies may be readily prepared using known procedures to produce hybridoma cell lines expressing antibodies to peptides of the invention. Such monoclonals antibodies may also be humanized e.g., using further known procedures which incorporate mouse monoclonal antibody light chains from antibodies raised to the mutants of the present invention with human antibody heavy chains.

In a further aspect, the invention relates to a diagnostic agent or assay component which comprises a monoclonal antibody as defined above. Although in some cases when the diagnostic agent or assay component is to be employed in an agglutination assay in which solid particles to which the antibody is coupled agglutinate in the presence of a β4GalNAcT in the sample subjected to testing, no labeling of the monoclonal antibody is necessary, it is preferred for most purposes to provide the antibody with a label in order to detect bound antibody. In a double antibody (“sandwich”) assay, at least one of the antibodies may be provided with a label. Substances useful as labels in the present context may be selected from enzymes, fluorescers, radioactive isotopes and complexing agents such as biotin. In a preferred embodiment, the diagnostic agent comprises at least one antibody covalently or non-covalently bonded coupled to a solid support. This may be used in a double antibody assay in which case the antibody coupled to the solid support is not labeled. The solid support may be selected from a plastic, e.g. latex, polystyrene, polyvinylchloride, nylon, polyvinylidene difluoride, cellulose, e.g. nitrocellulose and magnetic carrier particles such as iron particle coated with polystyrene.

The monoclonal antibody of the invention may be used in a method of determining the presence of β4GalNAcT in a sample, the method comprising incubating the sample with a monoclonal antibody as described above and detecting the presence of bound toxin resulting from said incubation. The antibody may be provided with a label as explained above and/or may be bound to a solid support as exemplified above.

In a preferred embodiment of the method, a sample desired to be tested for the presence of β4GalNAcT is incubated with a first monoclonal antibody coupled to a solid support and subsequently with a second monoclonal or polyclonal antibody provided with a label. In an alternative embodiment (a so-called competitive binding assay), the sample may be incubated with a monoclonal antibody coupled to a solid support and simultaneously or subsequently with a labeled β4GalNAcT competing for binding sites on the antibody with any toxin present in the sample. The sample subjected to the present method may be any sample suspected of containing a β4GalNAcT. Thus, the sample may be selected from bacterial suspensions, bacterial extracts, culture supernatants, animal body fluids (e.g. serum, colostrum or nasal mucous) and intermediate or final vaccine products.

Apart from the diagnostic use of the monoclonal antibody of the invention, it is contemplated to utilize a well-known ability of certain monoclonal antibodies to inhibit or block the activity of biologically active antigens by incorporating the monoclonal antibody in a composition for the passive immunization of a subject against diseases involving β4GalNAcT, which comprises a monoclonal antibody as described above and a suitable carrier or vehicle. The composition may be prepared by combining a therapeutically effective amount of the antibody or fragment thereof with a suitable carrier or vehicle. Examples of suitable carriers and vehicles may be the ones discussed above in connection with the vaccine of the invention. It is contemplated that a β4GalNAcT-specific antibody may be used for prophylactic or therapeutic treatment of a subject having a disorder involving β4GalNAcT.

A further use of the monoclonal antibody of the invention is in a method of isolating a β4GalNAcT, the method comprising adsorbing a biological material containing said enzyme to a matrix comprising an immobilized monoclonal antibody as described above, eluting said enzyme, from said matrix and recovering said enzyme from the eluate. The matrix may be composed of any suitable material usually employed for affinity chromatographic purposes such as agarose, dextran, controlled pore glass, DEAE cellulose, optionally activated by means of CNBr, divinylsulphone, etc. in a manner known per se.

In a still further aspect, the present invention relates to a method of determining the presence of antibodies against β4GalNAcT in a sample, the method comprising incubating the sample with β4GalNAcT and detecting the presence of bound antibody resulting from incubation. A diagnostic agent comprising the enzyme used in this method may otherwise exhibit any of the features described above for diagnostic agents comprising the monoclonal antibody and be used in similar detection methods although these will detect bound antibody rather than bound enzyme as such. The diagnostic agent may be useful, for instance as a reference standard or to detect β4GalNAcT antibodies in body fluids, e.g., serum, colostrum or nasal mucous, from subjects.

The monoclonal antibody of the invention may be used in a method of determining the presence of a β4GalNAcT, in a sample, the method comprising incubating the sample with a monoclonal antibody and detecting the presence of β4GalNAcT resulting from said incubation.

The present invention further contemplates, as noted elsewhere herein, a nucleic acid variant encoding β4GalNAcT as described herein wherein the nucleic acid sequence is a cDNA similar to a cDNA which encodes native β4GalNAcT, but differs therefrom in having one or more substituted codons or nucleotides which encodes the one or more substituted amino acids in the β4GalNAcT variant, as defined elsewhere herein, and wherein the substituted codon is any codon known to encode the substitute amino acid residue. The β4GalNAcT variant described herein may be produced by well-known recombinant methods using cDNA encoding the variant, the cDNA having been transfected or transposed into a host cell via a plasmid or other vector.

It is clear from the above that the present invention provides compositions and methods for the production of β4GalNAcT or active variants thereof, or cDNA which encode said proteins.

The invention further contemplates a method of making a hybridoma which secretes an antibody against β4GalNAcT or a variant thereof, comprising fusing a lymphocyte from an animal immunized with β4GalNAcT or a variant thereof with cells capable of replicating indefinitely in cell culture to produce the hybridoma and isolating the hybridoma.

All publications, patent applications, and patents mentioned herein are hereby expressly incorporated herein by reference in their entireties.

The abbreviations used are: LN or LacNAc, Galβ4GlcNAc; β4GalT, UDPGal: GlcNAcβ-R β1,4galactosyltransferase; LDN or LacdiNAc, GalNAcβ4GlcNAc; β4GalNAcT, UDPGalNAc: GlcNAcβ-R β1,4N-acetylgalactosaminyltransferase; pNP, 4-nitrophenyl; CHO, Chinese hamster ovary; HPAEC-PAD, high-pH anion-exchange chromatography with pulsed amperometric detection.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims. It is also to be understood that all base pair sizes given for nucleotides are approximate and are used as examples for the purpose of description.

Changes may be made in the construction and the operation of the various compositions and elements described herein or in the steps or the sequence of steps of the methods described herein without departing from the spirit and scope of the invention as defined in the following claims.

### Cited References

1. Figdor, C. G., van Kooyk, Y., and Adema, G. J. (2002) Nature Rev Immunol 2, 77-84

2. Dodd, R. B., and Drickamer, K. (2001) Glycobiology 11, 71R-79R

3. Leffler, H. (2001) Results Probl Cell Differ 33, 57-83

4. Angata, T., Kerr, S. C., Greaves, D. R., Varki, N. M., Crocker, P. R., and Varki, A. (2002) J Biol Chem

5. Amado, M., Almeida, R., Schwientek, T., and Clausen, H. (1999) Biochim Biophys Acta 1473, 35-53

6. Smith, P. L., Bousfield, G. R., Kumar, S., Fiete, D., and Baenziger, J. U. (1993) J Biol Chem 268, 795-802

7. Fiete, D., Beranek, M. C., and Baenziger, J. U. (1997) Proc Natl Acad Sci USA 94, 11256-11261

8. Yan, S. B., Chao, Y. B., and van Halbeek, H. (1993) Glycobiology 3, 597-608

9. Van den Nieuwenhof, I. M., Koistinen, H., Easton, R. L., Koistinen, R., Kamarainen, M., Morris, H. R., Van Die, I., Seppala, M., Dell, A., and Van den Eijnden, D. H. (2000) Eur J Biochem 267, 4753-4762

10. Van den Eijnden, D. H., Bakker, H., Neeleman, A. P., Van den Nieuwenhof, I. M., and Van Die, I. (1997) Biochem Soc Trans 25, 887-893

11. Do, K. Y., Do, S. I., and Cummings, R. D. (1997) Glycobiology 7, 183-194

12. van Remoortere, A., van Dam, G. J., Hokke, C. H., van den Eijnden, D. H., van Die, I., and Deelder, A. M. (2001) Infect Immun 69, 2396-2401

13. Nyame, K., Smith, D. F., Damian, R. T., and Cummings, R. D. (1989) J Biol Chem 264, 3235-3243

14. Srivatsan, J., Smith, D. F., and Cummings, R. D. (1992) Glycobiology 2, 445-452

15. Kang, S., Cummings, R. D., and McCall, J. W. (1993) J Parasitol 79, 815-828

16. Nyame, A. K., Leppanen, A. M., DeBose-Boyd, R., and Cummings, R. D. (1999) Glycobiology 9, 1029-1035

17. Nyame, A. K., Leppanen, A. M., Bogitsh, B. J., and Cummings, R. D. (2000) Exp Parasitol 96, 202-212

18. Powell, J. T., and Brew, K. (1976) J Biol Chem 251, 3653-3663

19. Powell, J. T., and Brew, K. (1976) J Biol Chem 251, 3645-3652

20. Shaper, N. L., Shaper, J. H., Meuth, J. L., Fox, J. L., Chang, H., Kirsch, I. R., and Hollis, G. F. (1986) Proc Natl Acad Sci USA 83, 1573-1577

21. Ramakrishnan, B., and Qasba, P. K. (2002) J Biol Chem

22. Ramakrishnan, B., and Qasba, P. K. (2001) J Mol Biol 310, 205-218

23. Asano, M., Furukawa, K., Kido, M., Matsumoto, S., Umesaki, Y., Kochibe, N., and Iwakura, Y. (1997) Embo J 16, 1850-1857

24. Lu, Q., Hasty, P., and Shur, B. D. (1997) Dev Biol 181, 257-267

25. Kotani, N., Asano, M., Iwakura, Y., and Takasaki, S. (2001) Biochem J 357, 827-834

26. Gastinel, L. N., Cambillau, C., and Bourne, Y. (1999) Embo J 18, 3546-3557

27. Almeida, R., Amado, M., David, L., Levery, S. B., Holmes, E. H., Merkx, G., van Kessel, A. G., Rygaard, E., Hassan, H., Bennett, E., and Clausen, H. (1997) J Biol Chem 272, 31979-31991

28. Sato, T., Furukawa, K., Bakker, H., Van den Eijnden, D. H., and Van Die, I. (1998) Proc Natl Acad Sci USA 95, 472-477

29. Nomura, T., Takizawa, M., Aoki, J., Arai, H., Inoue, K., Wakisaka, E., Yoshizuka, N., Imokawa, G., Dohmae, N., Takio, K., Hattori, M., and Matsuo, N. (1998) J Biol Chem 273, 13570-13577

30. Lo, N. W., Shaper, J. H., Pevsner, I., and Shaper, N. L. (1998) Glycobiology 8, 517-526

31. van Die, I., van Tetering, A., Schiphorst, W. E., Sato, T., Furukawa, K., and van den Eijnden, D. H. (1999) FEBS Lett 450, 52-56

32. Almeida, R., Levery, S. B., Mandel, U., Kresse, H., Schwientek, T., Bennett, E. P., and Clausen, H. (1999) J Biol Chem 274, 26165-26171

33. Guo, S., Sato, T., Shirane, K., and Furukawa, K. (2001) Glycobiology 11, 813-820

34. Lee, J., Sundaram, S., Shaper, N. L., Raju, T. S., and Stanley, P. (2001) J Biol Chem 276, 13924-13934

35. Nakamura, N., Yamakawa, N., Sato, T., Tojo, H., Tachi, C., and Furukawa, K. (2001) J Neurochem 76, 29-38

36. Bakker, H., Agterberg, M., Van Tetering, A., Koeleman, C. A., Van den Eijnden, D. H., and Van Die, I. (1994) J Biol Chem 269, 30326-30333

37. Bakker, H., Schoenmakers, P. S., Koeleman, C. A., Joziasse, D. H., van Die, I., and van den Eijnden, D. H. (1997) Glycobiology 7, 539-548

38. Van den Nieuwenhof, I. M., Schiphorst, W. E., Van Die, I., and Van den Eijnden, D. H. (1999) Glycobiology 9, 115-123

39. Neeleman, A. P., van der Knaap, W. P., and van den Eijnden, D. H. (1994) Glycobiology 4, 641-651

40. van Die, I., van Tetering, A., Bakker, H., van den Eijnden, D. H., and Joziasse, D. H. (1996) Glycobiology 6, 157-164

41. Smith, P. L., and Baenziger, J. U. (1988) Science 242, 930-933

42. Herman, T., and Horvitz, H. R. (1999) Proc Natl Acad Sci USA 96, 974-979

43. Okajima, T., Yoshida, K., Kondo, T., and Furukawa, K. (1999) J Biol Chem 274, 22915-22918

44. Easton, E. W., Blokland, I., Geldof, A. A., Rao, B. R., and van den Eijnden, D. H. (1992) FEBS Lett 308, 46-49

45. Palcic, M. M., Heerze, L. D., Pierce, M., and Hindsgaul, O. (1988) Glycoconj J 5, 49-63

46. Wiggins, C. A., and Munro, S. (1998) Proc Natl Acad Sci USA 95, 7945-4750.

47. Deutscher, S. L., and Hirschberg, C. B. (1986) J Biol Chem 261, 96-100

48. Stanley, P., and Siminovitch, L. (1977) Somatic Cell Genet 3, 391-405

49. Do, S. I., and Cummings, R. D. (1992) J Biochem Biophys Methods 24, 153-b 165.

50. Nagayama, Y., Namba, H., Yokoyama, N., Yamashita, S., and Niwa, M. (1998) J Biol Chem 273, 33423-33428

51. Brew, K., Vanaman, T. C., and Hill, R. L. (1968) Proc Natl Acad Sci USA 59, 491-497

52. Vliegenthart, J. F., Dorland, L., and van Halbeek, H. (1983) Adv Carbohydr Chem Biochem 41, 209-374

53. Deutscher, S. L., Nuwayhid, N., Stanley, P., Briles, E. I., and Hirschberg, C. B. (1984) Cell 39, 295-299

54. Sasaki, H., Bothner, B., Dell, A., and Fukuda, M. (1987) J Biol Chem 262, 12059-12076

55. Bierhuizen, M. F., and Fukuda, M. (1992) Proc Natl Acad Sci USA 89,9326-9330.

56. Manzella, S. M., Hooper, L. V., and Baenziger, J. U. (1996) J Biol Chem 271, 12117-12120

57. Saarinen, J., Welgus, H. G., Flizar, C. A., Kalkkinen, N., and Helin, J. (1999) Eur J Biochem 259, 829-840

58. Bergwerff, A. A., Thomas-Oates, J. E., van Oostrum, J., Kamerling, J. P., and Vliegenthart, J. F. (1992) FEBS Lett 314, 389-394

59. Dell, A., Morris, H. R., Easton, R. L., Panico, M., Patankar, M., Oehniger, S., Koistinen, R., Koistinen, H., Seppala, M., and Clark, G. F. (1995) J Biol Chem 270, 24116-24126

60. Smith, P. L., and Baenziger, J. U. (1990) Proc Natl Acad Sci USA 87, 7275-7279.

61. Smith, P. L., and Baenziger, J. U. (1992) Proc Natl Acad Sci USA 89, 329-333.

62. Dharmesh, S. M., Skelton, T. P., and Baenziger, J. U. (1993) J Biol Chem 268, 17096-17102

63. Mengeling, B. J., Manzella, S. M., and Baenziger, J. U. (1995) Proc Natl Acad Sci USA 92, 502-506

64. Green, E. D., Gruenebaum, J., Bielinska, M., Baenziger, J. U., and Boime, I. (1984) Proc Natl Acad Sci USA 81, 5320-5324

65. Green, E. D., Morishima, C., Boime, I., and Baenziger, J. U. (1985) Proc Natl Acad Sci USA 82, 7850-7854

66. Xia, G., Evers, M. R., Kang, H. G., Schachner, M., and Baenziger, J. U. (2000) J Biol Chem 275, 38402-38409

67. Fiete, D., Srivastava, V., Hindsgaul, O., and Baenziger, J. U. (1991) Cell 67, 1103-1110

68. Manzella, S. M., Dharmesh, S. M., Beranek, M. C., Swanson, P., and Baenziger, J. U. (1995) J Biol Chem 270, 21665-21671

69. Baenziger, J. U., Kumar, S., Brodbeck, R. M., Smith, P. L., and Beranek, M. C. (1992) Proc Natl Acad Sci USA 89, 334-338

70. Mulder, H., Spronk, B. A., Schachter, H., Neeleman, A. P., van den Eijnden, D. H., De Jong-Brink, M., Kamerling, J. P., and Vliegenthart, J. F. (1995) Eur J Biochem 227, 175-185

71. Neeleman, A. P., and van de Eijnden, D. H. (1996) Proc Natl Acad Sci USA 93, 10111-10116

72. Srivatsan, J., Smith, D. F., and Cummings, R. D. (1994) J Parasitol 80, 884-890.

73. Morelle, W., Haslam, S. M., Olivier, V., Appleton, J. A., Morris, H. R., and Dell, A. (2000) Glycobiology 10, 941-950

74. Do, K. Y., Do, S. I., and Cummings, R. D. (1995) J Biol Chem 270, 18447-18451.

75. Guerardel, Y., Balanzino, L., Maes, E., Leroy, Y., Coddeville, B., Oriol, R., and Strecker, G. (2001) Biochem J 357, 167-182

76. Hinnen et al., PNAS USA 75:1929-1933, 1978

77. Sambrook et al., Molecular Cloning: A Laboratory Manual 2nd Ed., Cold Spring Harbor Laboratory Press, 1989

78. Davis, L., Dibner, M. Battey, I., Basic Methods in Molecular Biology, (1986)

79. Gluzman (Cell, 23:175 (1981))

80. Kohler and Milstein, 1975, Nature, 256:495-497

81. Kozbor et al., 1983, Immunology Today 4:72

82. Cole, et al., 1985, in Monoclonal Antibodies and Cancer Therapy, Alan R. Liss, Inc., pp. 77-96

83. Inman, Methods in Enzymology, Vol. 34, Affinity Techniques, Enzyme Purification Part B, Jacoby and Wichek (eds) Academic Press, New York, P. 30, 1974

84. Wilcheck and Bayer, The Avidin-Biotin Complex in Bioanalytical Applications Anal. Biochem. 171:1-32, 1988

85. Hutchinson, C., et al., 1978, J. Biol. Chem. 253:6551

86. Zoller and Smith, 1984, DNA 3:479-488

87. Oliphant et al., 1986, Gene 44:177

88. Hutchinson et al., 1986, Proc. Natl. Acad. Sci. U.S.A. 83:710

89. Higuchi, 1989, “Using PCR to Engineer DNA”, in PCR Technology: Principles and Applications for DNA amplification, H. Erlich, ed., Stockton Press, Chapter 6, pp. 61-70

90. D. Shortle et al. (1981) Ann. Rev. Genet. 15:265

91. M. Smith (1985) ibid. 19:423

92. D. Botstein and D. Shortle (1985) Science 229:1193

93. S. McKnight and R. Kingsbury (1982) Science 217:316

94. R. Myers et al. (1986) Science 232:613

95. Good et al., Nucl. Acid Res 4:2157, 1977

96. Conolly, B. A. Nucleic Acids Res. 15:15(8\7): 3131, 1987

97. Innis et al., Academic Pres, 1990

98. M. A. Innis and D. H. Gelfand, PCR Protocols, A Guide to Methods and Applications, M. A. Innis, D. H. Gelfand, J. J. Shinsky and T. J. White eds, pp 3-12, Academic Press 1989

99. Barney in “PCR Methods and Applications”, Aug 1991, Vol 1(1), page 4, and European Published Application No. 0320308, published Jun. 14, 1989

100. Harlow, E. et al., Antibodies. A Laboratory Manual, Cold Spring Harbor Laboratory Press, Cold Spring Harbor, N.Y. (1988)

101. Thompson, J. D. et al (1994) Nucleic Acids Res 22:4673

What is claimed is:

1. A purified β4 acetylgalactosaminyl transferase which is substantially free of other proteins.

2. The purified β4 acetylgalactosaminyl transferase of claim 1 having SEQ ID NO: 1.

3. A purified β4 acetylgalactosaminyl transferase which is substantially free of other proteins, comprising an amino acid sequence which has at least about 90% identity with SEQ ID NO: 1, and which has enzymatic activity of a β4 acetylgalactosaminyl transferase.

4. A recombinant β4 acetylgalactosaminyl transferase comprising SEQ ID NO: 1.

5. An isolated polynucleotide which encodes a protein having β4 acetylgalactosaminyl transferase activity and which is selected from the group consisting of:
(A) a polynucleotide which selected from the group consisting of SEQ ID NO:2 and an expressible coding sequence of SEQ ID NO:2;
(B) a polynucleotide which differs in nucleotide sequence from the polynucleotides of (A) above due to degeneracy of the genetic code and which encodes a protein having β4 acetylgalactosaminyl transferase activity; and
(C) a polynucleotide which differs in nucleotide sequence from the polynucleotides of (A) or (B) in that said polynucleotide lacks a nucleotide sequence which encodes a transmembrane domain wherein the β4 acetylgalactosaminyl transferase encoded is soluble.

6. The polynucleotide of claim 5 wherein the polynucleotide is DNA.

7. A vector containing the polynucleotide of claim 5.

8. A host cell transformed or transfected with the vector of claim 7.

9. A process for producing a protein having β4 acetylgalactosaminyl transferase activity comprising the steps of:
culturing the host cell of claim 8 thereby expressing the β4 acetylgalactosaminyl transferase; and
purifying the 64 acetylgalactosaminyl transferase from the cultured host cell.

10. The process of claim 9 wherein the protein having β4 acetylgalactosaminyl transferase activity is soluble.

11. The host cell of claim 8 wherein the polynucleotide is operatively associated with an expression control sequence contained in said vector.

12. The host cell of claim 8 transformed or transfected with an expressible polynucleotide encoding a peptide or polypeptide requiring post-translational formation of an LDN structure thereon.

13. An isolated polynucleotide which encodes a protein having β4GalNAcT activity and which is selected from the group consisting of:
(A) a polynucleotide which hybridizes with a nucleic acid selected from the group consisting of SEQ ID NO:2 or an expressible coding sequence thereof;
(B) a polynucleotide which hybridizes with a nucleic acid which differs in nucleotide sequence from the isolated polynucleotides of (A) above due to degeneracy of the genetic code and which encodes a protein having β4GalNAcT activity; and
wherein the polynucleotides of (A) and (B) hybridize under stringency conditions comprising prehybridization and hybridization at 68° C. followed by washing twice with two×SSC, 0.1% SDS at 22° C., and washing twice with 0.2×SSC, 0.1% SDS at 22° C.; or prehybridization and hybridization at 42° C. in 5×SSPE, 0.3% SDS, 200 ug/ml sheared and denatured salmon sperm DNA, and 25% formamide, or 35% formamide, or 50% formamide, and washing with 2×SSC, 0.2% SDS at 50° C.

14. The polynucleotide of claim 1 wherein the polynucleotide is DNA.

15. A vector containing the polynucleotide of claim 13.

16. A host cell comprising the vector of claim 15.

17. A method for producing a protein or peptide having a GalNAcβ1,4 GlcNAc structure thereon, comprising the steps of:
providing a host cell having an expressible polynucleotide encoding a peptide or polypeptide requiring a GalNAcβ1,4GlcNAc structure and transformed or transfected with the vector comprising a polynucleotide encoding a β4GalNAcT;
expressing in the host cell the β4GalNAcT and the protein or peptide requiring the GalNAcβ1,4 GlcNAc structure thereon thereby forming a glycosylated protein or peptide having the GalNAcβ1,4GlcNAc structure; and
purifying the protein or peptide having the GalNAcβ1,4GlcNAc structure thereon.

18. The method of claim 17 wherein the polynucleotide comprises SEQ ID NO: 2 or an expressible coding sequence thereof.

19. The method of claim 17 wherein the β4GalNAcT comprises SEQ ID NO: 1 or a variant thereof having β4GalcNAcT activity. 20. An in vitro method of producing a protein or peptide having a GalNAc β1,4GlcNAc structure thereon, comprising the steps of:
providing a protein or peptide requiring a GalNAcβ1,4GlcNAc structure;
providing a protein having β4GalNAcT activity;
providing a GalNAc donor; and
combining the protein or peptide requiring the GalNAc β1,4GlcNAc with the protein having β4GalNAcT activity, and with the GalNAc donor thereby forming a protein or peptide with the GalNAc β1,4 GlcNAc structure.

21. A monoclonal antibody raised against a β4GalNAcT protein or peptide.

22. The monoclonal antibody of claim 21 raised against SEQ ID NO: 1 or an antigenic portion thereof, wherein the monoclonal antibody binds specifically to SEQ ID NO: 1.
